(19) United States
(12) Patent Application Publication
(10) Pub. No.: US 2009/0254097 A1
(43) Pub. Date: Oct. 8, 2009

(54) SYSTEM AND DEVICE FOR DESIGNING AND FORMING A SURGICAL IMPLANT

(76) Inventor: Robert E. Isaacs, Chapel Hill, NC (US)

Correspondence Address: PASSE' INTELLECTUAL PROPERTY, LLC, 1717 BRASSFIELD RD., RALEIGH, NC, 27614, US

(21) Appl. No.: 12/098,375

(22) Filed: Apr. 4, 2008

(51) Int. Cl. A61B 19/00; A61B 17/58

(52) U.S. Cl. 606/130; 606/102

(57) ABSTRACT

The present invention relates to a method of determining the proper shape of a surgical linking device that is to be attached to a bony body structure such as the spinal column. The present invention also relates to methods of shaping and bending surgical linking devices such that they fit attachment means or are shaped to fit the bony body structure.

### COPYRIGHT NOTICE

A portion of the disclosure of this patent contains material that is subject to copyright protection. The copyright owner has no objection to the reproduction by anyone of the patent document or the patent disclosure as it appears in the Patent and Trademark Office patent files or records, but otherwise reserves all copyright rights whatsoever.

### BACKGROUND OF THE INVENTION

1. Field of the Invention

The present invention is directed to systems and devices related to the design and formation of surgical implants such as surgical linking devices. More particularly the present invention provides a system and devices for forming or shaping a surgical implant to conform to two or more selected attachment points (including surface anatomy) in a six degree of freedom method for attachment.

2. Description of Related Art

Fixation systems for aligning, adjusting and or fixing, either partially or rigidly, portions of a patient's bony anatomy in a desired spatial relationship relative to each other is frequently used in orthopedic type surgery. For example, in spinal related surgery for repair or positional adjustment of the vertebrae, it is often necessary that multiple vertebrae are surgically manipulated. As spinal surgery often requires the instrumentation of more bony elements than other areas of orthopedic surgery, the linkage devices can be extremely challenging to design and implant. Medical states such as scoliosis, spinal injury, disk problems and the like make use of spinal rod fixation systems for positioning the vertebrae and permanently supporting the spinal relationship for the purposes of treatment of the various conditions.

A spinal rod needs to be oriented in six degrees of freedom to compensate for the anatomical structure of the particular patient's spine and the particular attachment points or methods for attaching the rods to the vertebrae. In addition, the physiological problem being treated as well as physician's preferences will determine the exact configuration necessary. Accordingly, the size, length and particular bends of each spinal rod depends on the size, number and position of each vertebra to be held, their spatial relationship as well as the fixating means, such as pedicle screws, used to hold the rods attached to each vertebra. The relationship of the vertebrae will be different for each patient and the positioning of the patient at the point of installation of the rods. During surgery, the orientation of the spine and vertebrae can be very different than the corresponding position of a patient's upright posture. Rods are bent in one or more anatomic planes measured by distance from each bend, angle of the bend and rotation in relationship to other bend points in order to fit into two or more vertebral anchors.

The bending of a spinal rod can be accomplished by a number of methods. The oldest and most widely used method for bending rods manually during surgery is a three point bender called a French Bender in which a bending pliers type device is manually operated to place one or more bends in a rod. The French Bender requires both hands to operate and provides leverage based on the length of the handle. While the device can make it relatively easy to bend a spinal rod, the determination of the location, angle and rotation of bends using such a device is relatively arbitrary. As it is arbitrary, even further problems can occur from bending a device and then rebending to fix mistakes which impose metal fatigue into a rod thus increasing the risk of a mechanical failure. Even further stress risers can be imposed and increased time in the operating room (OR) adds additional morbidity to surgery. Spinal rods are usually titanium or other extremely hard metal and as such are difficult to bend without some sort of leverage based bender. In addition, since several spatial relationships have to be maintained in using a French Bender, the process, which usually occurs during surgery, can take an extremely long time and its use requires a great degree of physician skill to accomplish an accurate final product. Even still it is difficult to get well shaped rod using the French Bender. Accordingly, the art has attempted various ways to overcome the limitations of the currently preferred technology.

A number of manual benders are described in the art. In U.S. Pat. No. 5,113,685 issued May 19, 1992 to Asher et al, there is described an apparatus for use in bending rods and plates to the spinal column comprising an elongated bar with a variety of bending angles for bending more angles than the French Bender. However, this device is hard to use and provides no means for determining the six degrees of spatial relationship that each bend must make. In US patent application 2006/0150699 published Jul. 13, 2006 to Garner, et al, there is an instrument and method for bending rod using a lever pliers type device having bearing surfaces. In addition, the angle of bend can be determined by use of a gauge that indicates angle bend by degree of grip movement. While useful in being easier to use, it does not aid in determination of the other degrees of freedom either in calculating them or in making the final bends.

An automatic method designed for pre-surgical formation of spinal rods is disclosed in US patent application 2005/0262911 published Dec. 1, 2005 to Dankowicz, et al. An automatic series of shaping steps is “imposed” on a rod from an input mechanism for producing the desired multi-dimensional bent shape. The unfortunate problem with the device is it relies on a pre-surgical determination of the points at which bends occur to determine the final shape of the rod. While it is possible to anticipate where the anchors might ideally end up and occasionally be correct, surgery implantation of attachment points is as much of an art as science and a pre-formed rod may not be accurately produced when compared to the anchor means as they are actually installed in the spine. This leads to a catastrophic condition where the surgeon has the patient laid open with a rod that does not fit the attachment points. In addition the disclosure implies the device is unsuitable for use during surgery. Even further the device is relatively large and those in the art still would prefer a manual means of producing a rod during surgery because of the ability to make minute adjustments based on feedback during surgery.

It appears that the art has been toying with the idea of computer aided design but has not accomplished anything due to the lack of bending devices as well as a lack of understanding of all the issues involved in bending surgical devices. In, “A pilot study on computer-assisted optimal contouring of orthopedic fixation devices,” Comput Aided Surg. 1999; 4 (6):305-13, indicated that overcoming these problems would be difficult if not impossible.

Image guided surgical systems, for example, devices produced by BrainLAB, as well as three dimensional digitizers are already in the art and some are already FDA approved for use during surgery. These devices are fairly commonly used by some physicians in the operating environment. By moving the digitizer through space or inputting a particular point in space a map can be produced of spatial relationships. In U.S. Pat. No. 6,400,131 issued Dec. 31, 2002 to Leitner et al, there is described a contour mapping system applicable as a spine analyzer and probe. It is disclosed as useful to determine the curvature of the spine while standing and contour mapping of the spine in the intact (non-surgical) patient.

Accordingly, a means for designing and forming a surgical linking device, especially for linking bony parts of the body, for use in a surgical orthopedic procedure such as the attachment of a spinal rod, that is accurate, quick and takes the various input characteristics into account for the specific implanted device as actually needed would be of great value during an orthopedic implant surgery such as spinal surgery.

### BRIEF SUMMARY OF THE INVENTION

In one embodiment there is a system for shaping a surgical linking device for attachment to a selected bony body structure having at least two linking device attachment means comprising:
a) a means for determining the relative spatial location of at least one of the attachment means and the bony structure;
b) a means for converting the relative spatial location into a digital format;
c) a computer capable of receiving the digital format in b) and using the relative spatial location to determine one or more shape locations in the surgical linking device, each shape location having one or more of a shape angle and shape rotation at each one or more shape locations such that shaping of the surgical linking device will enable the surgical linking device to attach to the bony body structure using the attachment means; and
d) a means for delivering the determined shape information to a computer output.

In yet another embodiment there is a surgical linking device on a selected bony body structure comprising:
a) placing at least two linking device attachment means on the bony body structure at desired locations;
b) digitally determining the relative spatial location of at least one of the bony structure and the attachment means;
c) transferring the digitized information to a computer which determines information of one or more of
   i. one or more of the location, angle and rotation of shapes in a selected surgical linking device that could be made in order for the linking device to be attached to the bony structure using the attachment means;
   ii. one or more adjustments to the position of or addition to the attachment means that could be made so that a selected preformed, partially preformed or a minimally shaped surgical linking device can be attached to the bony structure with the attachment means;
d) delivering the computer determined information to a computer output;
e) using the information from the computer output to perform one or more of:
   i. selecting a preformed or partially preformed surgical linking device;
   ii. shaping a surgical linking device with a device that measures one or more of the shape location, shape angle and shape rotation; and
   iii. adjusting the position of or adding to the attachment means; and
f) attaching the surgical linking device to the attachment means.

Yet another embodiment includes a device for manually bending a surgical linking device comprising:
a) a manually operated lever for bending the linking device; and
b) at least 2 bend measuring means selected from the group comprising, bend position measuring means, bend angle measuring means and bend rotation measuring means.

Another embodiment of the invention includes a device for determining the rotation for placing a bend in a surgical linking device comprising:
a) a circular gauge indicating the degrees of rotation; and
b) a means for positioning the device on the surgical linking device or on a means for bending the linking device such that the gauge aligns with any bends in the linking device.

Yet still another embodiment is a means for determining the selection of a preformed surgical linking device for use in attaching to a selected bony body structure having at least two linking device attachment means comprising:
a) a means for determining the relative spatial location of each attachment means;
b) a means for converting the relative spatial location into a digital format;
c) a plurality of preformed surgical linking devices;
d) a computer having selected spatial information about the preformed linking devices wherein the computer is capable of receiving the digital format in b) and using the digital format to determine if one of the preformed surgical linking devices fits the attachment means and if there is none that fit, if one or more attachment means could be adjusted in relative location such that one of the preformed surgical linking devices could be selected and fit the attachment means; and
e) a means for delivering the determined attachment means adjustments and selected preformed linking device to a computer output.

One more embodiment includes a method for placing multiple bends with 6 degrees of freedom in a surgical linking device comprising:
a) establishing a starting point on the device;
b) holding the device relative to the starting point;
c) moving the device and measuring away from the starting point to establish a second point on the device for placing a bend with 6 degrees of freedom; and
d) repeating steps b) and c) using either the starting point or the second point to hold from until the multiple bends are completed.

Another embodiment of the present invention is a process for producing one or more shapes in a surgical linking device comprising:
a) a digital process for determining the desired spatial parameters of the shapes to be produced; and
b) a manual shaping process linked to the digital process wherein the shaping process applies the spatial parameters to the surgical attachment device.

### BRIEF DESCRIPTION OF THE DRAWINGS

FIGS. 1a through 1d depict a surgical rod and various bends with 6 degrees of freedom.

FIG. 2 depicts three vertebrae each with a surgical rod attachment screw.

FIG. 3 depicts three vertebrae with a bent surgical rod attached to the three rod attachment screws.

FIG. 4 depicts a front view of a rotation gauge for attaching to a surgical rod.

FIGS. 5a and 5b depict surgical rods with ruled markings.

FIG. 6 depicts a small hand device for bending a surgical rod and having a means for measuring location, rotation and angle bend.

FIG. 7 is a perspective view of a dual lever surgical rod bending device.

FIG. 8 is a side view of a dual lever surgical rod bending device.

FIG. 9 is a view of a dual lever surgical rod bending device with the levers in the open position.

FIG. 10 is an end on perspective which allows view of the fulcrum means.

FIG. 11 is a flow diagram of an embodiment for determining bend information.

### DETAILED DESCRIPTION OF THE INVENTION

The present invention refers to a method for improving the shaping of a surgical linking device, for example, by bending. First, by digitally calculating appropriate shapes such as bends in 6 degrees of freedom (three dimensional) and then outputting that information to the surgeon or other medical personnel or to a bending device, a linking device can easily and quickly be shaped by casting, bending or the like. Second, is disclosed a device for quickly and easily taking the input from a digitally calculated means or other means and manually shape a precisely bent or shaped linking device. Accordingly, the time spent in surgery bending linking devices can be greatly reduced thus improving the chances of a successful operation without complications as well as reduce the cost of such an operation, for example, from rebending or bending a second device. Since a significant portion of time is spent in bending and in some cases rebending such devices, taking minutes to an hour or more off the time to bend a linking device correctly is an important advance in the art.

While this invention is susceptible of embodiment in many different forms, there is shown in the drawings and will herein be described in detail specific embodiments, with the understanding that the present disclosure of such embodiments is to be considered as an example of the principles and not intended to limit the invention to the specific embodiments shown and described. In the description below, like reference numerals are used to describe the same, similar or corresponding parts in the several views of the drawings. This detailed description defines the meaning of the terms used herein and specifically describes embodiments in order for those skilled in the art to practice the invention.

The terms “a” or “an”, as used herein, are defined as one as or more than one. The term “plurality”, as used herein, is defined as two as or more than two. The term “another”, as used herein, is defined as at least a second or more. The terms “including” and/or “having”, as used herein, are defined as comprising (i.e., open language). The term “coupled”, as used herein, is defined as connected, although not necessarily directly, and not necessarily mechanically.

Reference throughout this document to “one embodiment”, “certain embodiments”, “and an embodiment” or similar terms means that a particular feature, structure, or characteristic described in connection with the embodiment is included in at least one embodiment of the present invention. Thus, the appearances of such phrases or in various places throughout this specification are not necessarily all referring to the same embodiment. Furthermore, the particular features, structures, or characteristics may be combined in any suitable manner in one or more embodiments without limitation.

The term “or” as used herein is to be interpreted as an inclusive or meaning any one or any combination. Therefore, “A, B or C” means “any of the following: A; B; C; A and B; A and C; B and C; A, B and C”. An exception to this definition will occur only when a combination of elements, functions, steps or acts are in some way inherently mutually exclusive.

As used herein the term “bending” refers the act of forcing or the like a linking device from a first position at a particular point to a second angular or curved position at that point in three dimensional space. The so-called 6 degrees of freedom is considered in bending a particular device once the location of the bend is determined. In general, once the position of the placement of a bend is determined, then the angle of the bend and in many cases the rotation off of a central axis may also be determined. In many cases a simple angular shaping is sufficient while in others, such as is often the case for surgical rods, a rotation off axis is necessary. The bending is exemplified in the drawings which explanation follows. As used herein “shaping” refers to not only bending but other methods of taking the 6 degrees of freedom information generated with the present invention and producing a shaped device. In addition to bending, the use of extrusion, casting, deformation, molding and the like could be considered a means of shaping a particular device with the information generated herein. See, for example, U.S. Pat. No. 6,749,614 issued Jun. 15, 2004 to Teitelbaum, et al. for an example of such material which could be used to shape a linking device with the present invention methods.

A “surgical linking device” as used herein refers to those devices used during surgery to use to bind to a selected bony body structure to mend, stabilize, move, reshape, correct deformities or strengthen such as attachments made to bones. For example, surgical rods, surgical plates, surgical transverse connecting rods, surgical wire or surgical cable and the like are used in surgery to mend, stabilize or correct breaks, correct deformities and the like in selected bones by attachment to 2 or more attachment points. Such plates and rods usually are supplied straight in a number of lengths or preformed arcs and must be bent to fit their intended use. (See v2-Evren 2008 online catalog, www.v2evren.com.tr for examples of vertebral rods and connectors as well as other orthopedic devices of a surgical nature). Typically, these devices are made of titanium or other extremely durable, stiff and difficult to bend material. Rigid materials such as titanium, commercially pure titanium, stainless steel, cobalt chrome and the like could be used. Other materials include flexible materials such as made of PEEK or other appropriate plastics, graphite or the like, bumpered systems and devices in both mono and multi diameter versions. Where casting or other shaping means are used, any rigid material suitable for surgical use in these conditions can be used. Additionally, useful are shape memory alloys, shape altering devices, materials with varying stiffness, biological materials and any synthetic material with bioactive properties.

The French Bender is the surgical instrument of choice today to bend these materials but it does so without regard to being able to measure the 6 degrees of freedom of movement in any manner. Accordingly, the process of bending a surgical rod with a French Bender is laborious, demanding, requires some degree of artistry and frequently requires starting over. (See the French Bender in V2 catalog, supra). Other surgical linking devices could include plates attached to specific body parts, both in the appendicular and axial skeleton. Also, items such as cables and rigid clamps used to affix to and alter teeth and their alignment.

A “linking device attachment means” refers to a means attached to a body structure designed to received the surgical linking device and hold it in place. Surgical clamps and screws are common examples of these devices. In the case of a surgical rod, a variety of surgical screws, bolts, and hooks are available to screw into the bone and or to hold the rods in place. These include polyaxial screws, mono axial screws fixed angle screws, iliac screws, sacral screws, lateral mass screws, bolts, laminar hooks and pedicle hooks. In additions, items such as staples, or plates that serve to hold one body part, can serve as an anchor to which a linking device can be affixed onto the spine especially with anterior plating systems. All these systems can be used together and further connect up to similar anchoring plates. Connectors such as axial, lateral and transverse connectors are used and frequently locking screws are used to hold the linking device in place. Even further, the attachment means could be devices added to the attachment means to change the attachment position. A screw attachment, for example, could be used. In the practice of this invention, the devices and methods of this invention anticipate use when there are at least 2 and frequently 3, 4 or more attachment means corresponding to each surgical linking device. One understands that multiple differing types of attachment means could be used in a single installation. In addition, in the case of plates, the attachment means may be installed after the shaping of the plate based on the shape of the plate and not the other way around. The claims anticipate this and are to be so interpreted.

“Determining the relative location of each attachment means and bony structure” refers to understanding the spatial relationship between the bony structure and any points of attachment so that a linking device such as a surgical rod can connect between the attachment points given the proper shape of the device. The relative location can be done with currently available image guidance devices such as three dimensional digitizers (such as the Polhemus Patriot) which can be used simply by engaging the device at several points such as the attachment points or along the bony structure and letting a computer in the device or elsewhere digitize the information. A partially manual method could be done, for example, by photographic means such as x-ray or regular photography and the spatial relationship determined away from the patient. Such method might need a plurality of photographs but given this explanation is well within the skill in the art. From the determination of the relative spatial location, the information can easily be digitized either automatically, as is the case with the three dimensional digitizer, or by entering hand calculated information into a computer or the like which then stores the information digitally. Either way, the information is converted into a digital format which a computer is capable of manipulating. Other devices could be optical, EM, image guidance systems, Shape Tape™, ultrasound, cat scans, and other radiographic devices. The key is that information needs to be gathered about spatial relationships and that information is capable of being obtained in a variety of ways. It is clear that the enumerated means or any other means which achieves the determination of the spatial relationship can be used by one skilled in the art. In some embodiments it also refers to making multiple determinations after adjustments to the installation or attachments means are made. One skilled in the art will know when to make multiple determinations or if one is enough.

Since structures such as the shape of the patient's anatomy, bone structure, other devices in the area and the like may also need to be considered when determining the bend profile, it is also one embodiment of the invention where that other structural information is also created in a digital format for transfer or use by a computer. In one embodiment, the contour or structure can serve as the input by itself, such as with any plating system, where the input is the topography of the surface of the body part, and this is how the implant is shaped. The attachment points are then driven through the plate after the plate is shaped, not prior, in as much as the information could be determined solely from surface anatomy and not the attachment points.

A computer such as a laptop, hand held device, desktop or other computer device can receive or possibly have received by the fact it converted hand data to a digital format, the relative location of the attachment means and/or the bony structure in a digital format. The computer then programmed with the spatial information can determine the best way to shape/bend or the like the linking device in order to fit the attachment means. This needs to also take into consideration the fact that other structures or the shape of the structure being attached to may be in the way and bends will take that into consideration as well. For example, when binding the vertebrae, the shape of the bones must also be considered in the bends used.

The computer can be programmed to accommodate any number of parameters in determining the output or the final shape of the linking device. In this way, the goals of surgery can be assisted in being obtained through the alteration of the shape of the linking device. Whereas in one embodiment, the shape dictated by the information above and not altered further could be used to create the linking device, further alterations in the device's shape can help to address, straighten, or alter abnormalities in alignment of the body part(s), create lessen or eliminate deformities, reduce or impose changes in alignment or the addition or elimination of stresses. It is possible to couple the changes in different planes or simply correct in one (and not, for example, the other orthogonal) plane. These modifications of the shaping information that is outputted can be obtained through various means—visual, anatomic, guided by radiographs (intraoperative, preoperative, positioning films, etc.), guided by the material properties of the linking device and the plasticity and/or relative location of the body part(s) being altered.

The computer would have no direct interaction with the device used for bending in one embodiment. In other embodiments, it could input the information directly to the shaping device such as to a screen or other means such as to set the dials prior to shaping. The computer defines mathematically from the spatial location of the attachment means and the bony structure of the body, the heads of screws, surface of that bony body part and the like, a curve which approaches these points in three dimensional space within the requirements and capabilities of the selected surgical linking device. The determined information can be used to select a specific device, to place bends in an unbent or pre-bent device (or shape as needed) or to adjust the attachment means as desired. In addition, a number of different shape solutions could be accommodated such that the surgeon can use personal judgment in selecting the best shape solution.

The computer could further customize the output of the bend information. It could minimize the number of bends if desired (for example, with a quicker zigzag type design with greater bend angles at fewer bend points but with potentially greater stress risers). In other embodiments it could increase the number of bend locations to create a smoother design, as the more bend points the smoother the bend. One could limit the program or the device for that matter to specific angles so that all angles would be above, at or below a particular value. It could also limit the choices to incremental choices such as every 5 degrees of bend or rotation or distances to a few millimeters. A simple design connecting points could be achieved as could a more complex design as desired. The computer could determine the size of the device, can determine if the attachments means can be adjusted or added to with offsetting devices (and therefore increase or decrease the number of bends to attach the points). In one embodiment, the program can be used to see if the attachment points can be used with a pre-bent device either without modification or with adjustment of the attachment means or the addition of spatial offsetting devices. The computer could also pick shapes that simplify the shape of the linking device or improve its biomechanics.

When bending a device the device can be in 4 or 6 degrees of freedom. First, to be determined is a bend location. The bend location is a point on the linking device where the bend will occur. It can be measured from a starting point, for example, 1.5 cm from the distal end of a surgical rod. Or can be determined by selecting from a set of fixed points on the device. So for example, ruled markings every centimeter on a rod or other device could be marked as point 1, 2; or 1 cm, 2 cm, etc and the output of the computer deliver the fixed point. In another embodiment, the device is held in place and moved a given distance from the point held as a reference starting point.

The bend angle is the degrees that the device is bent off of the horizontal. The bend can be accomplished as a single bend or it can be a multiplicity of bends as described above. In general, the bends will be from just greater than zero to 180 degrees off of straight. In many embodiments the bend angle is 90 degrees or less. In general, bend angle will be determined by a number of factors including the particular use, the surgeons desires and the like. In addition, the angle of rotation off of the direction the device was going could be determined. So, for example, a surgical rod could be angled from zero to 360 degrees off of the zero axis of the original direction of the rod in addition to the bend. This a bend of, for example, 45 degrees with a rotation of 15 degrees 2 centimeters from a starting point could define a particular bend output. The distance rotation and bend angle after determination is then delivered to a computer output. The output can be a paper output, a GUI (Graphic User Interface) or the like, such that a user can read the information and begin the process of bending a device. In one embodiment, the information is delivered directly to the bending device.

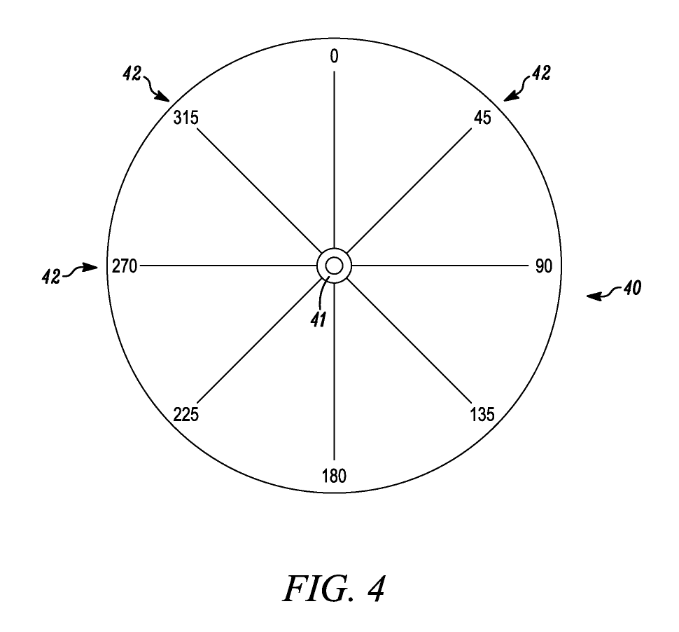

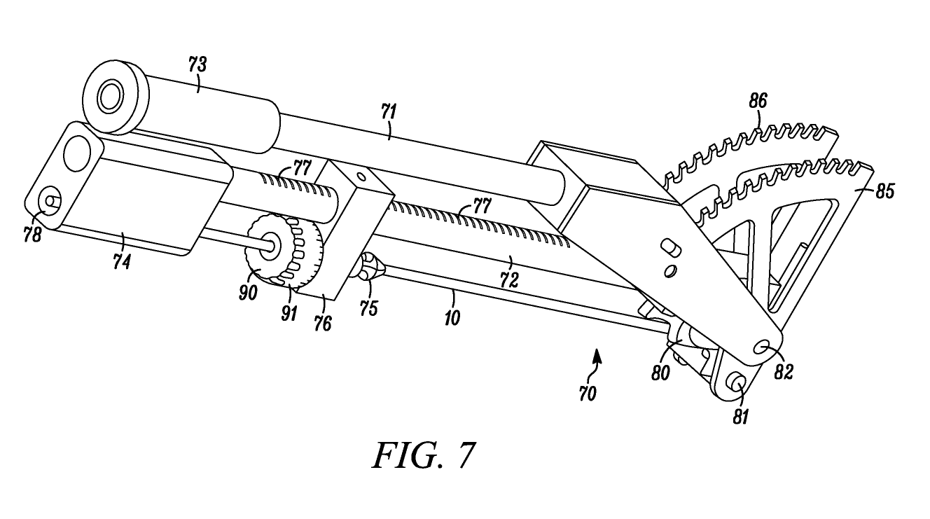

The means for placing a bend in the surgical linking device can, in one embodiment, be accomplished by one or more manual devices. Hand measuring distance, a rotation disk (as shown in FIG. 4), and then a bending device for bending to an angle could allow the bending with three interactive devices. Likewise, the device shown in FIG. 7 could be used to set all three parameters on one device. Where a device that only needs 4 degrees of freedom, the computer needs only produce distance and bend angle and the various devices above either singly or one single device could be used. Rotation in this case could be set at zero. Further, such as in the case wherein the output of the system determines that a pre-bent rod could be used, the output of all of the parameters except distance could be zero. The system could simply determine which linking device that should be chosen, with or without the need to further manipulate the screw locations or add additional offsetting devices. In this case, no bends may need to be made.

Surgically, the method for installing the surgical linking device on a body bony structure using the present invention, in one embodiment, could be started by placing at least two linking device attachment means on the body structure at desired locations. Then the spatial relationship of the attachment means could be determined in a digital manner. The digitized information would be transferred to (including calculated by) a computer which determines one or more of the following: one or more of the bend location, bend angle and bend rotation such that upon making the bends the device will fit the installed attachment means; it could also determine that one or more adjustments or additions to the position of the attachment means could be made so that one could select a preformed or partially preformed device or that a device could be bent with fewer bends or no bends at all to fit the attachment means. The computer calculates and delivers the information to a computer output. The output could be used to perform one or more functions during surgery, namely selecting a preformed or partially preformed surgical linking device; placing one or more bends as described above in the device or adjusting the position of the attachment means or placing an addition to the attachment means. After the proper selection and bending the surgical linking device is attached to the attachment means.

The advantages and uses of the computerized means for determining the shape of a surgical linking device are several. It allows for the facilitated implantation of preformed whole rods or segments, and the ability to define the size and shape of the component pieces of a multi-component linking device. The linking device can aid a surgeon in the formation of the desired end result rather than the situation as confronted. The linking device can be designed and formed based on the intersection of this desired end result, the current position of the anatomy, and the location of the affixing points. This can be used to control the reduction of fractures and deformities by defining the amount to translation, rotation and or angular correction and altering the shape of a linking device to achieve the result. Further, it can be used to correct spondylolisthesis. In another embodiment, this method could be used to define the resultant rod and thus help form, obtain and or hold the correction required in performing an osteotomy. The linkage device can be implanted without any static load imparted to the body, or with a predefined load, which can aid in adjusting deformities or set the location of a flexible system. One could determine how the anatomy moves or has moved or changed and one can determine the amount of implant manipulation needed to gain the anatomical change desired. (For example, using x-rays in the OR and compare to those taken prior to surgery to figure out how much of a bend one needs to achieve the straightness the patient can achieve by bending). As all people's anatomy changes to some degree when lying in an OR table versus the upright position, the present invention could be used to account for this change as well.

Although in one embodiment, the rod can be formed quickly at the time of surgery, this is not required. One could immediately implant or defer the linking device implantation such as to let ongrowth or ingrowth occur then implant the formed rod in a delayed fashion.

Further, this system also is ideal to custom design large percutaneous implants. As well, it could be used to design a crosslink that joins two or more linking devices or any other type of implant that could benefit from linking. Further, it can be used to accommodate an easy way to extend the linking device should this be required in the future, as the end configuration and angle of one embodiment of this device is know at the time of production and therefore this additional step (which is useful typically in a delayed fashion months to years later) could further be incorporated.

Bending is accomplished manually by means of known means but in the alternative can be accomplished with novel devices of the present invention. Novel bending devices all comprise at least one lever. By lever as used herein refers to a bar or long arm that can be used to bend an object around a particular pivot point. With one lever the object to be bent is forced with the aid of the lever. In other embodiments, there is a pair of levers that can bend around a fulcrum that is a point or device that will aid in bending the device around.

Devices such as the French Bender have no means for determining any of the bend parameters discussed above when bending a surgical linking device. The present bending device has a means for determining at least two of those parameters. In one embodiment, the two parameters are location and bend angle. In another embodiment, the device measures location, bend angle and bend rotation. Each lever can have a handle disposed at a distal end to aid in grabbing the lever and leveraging it during use.

The means to measure the spatial parameters can measure a continuous location or angle or in other embodiments the measurement means can measure incrementally (i.e. non-continuously). So, for example, the location can measure in half centimeter one centimeter or the like increments, the angle of bend or rotation could be measured in five degree increments or the like. Continuous measurement or click stop measurement could be used with each measuring means individually or mixed as desired. One desiring higher accuracy might want continuous rather than incremental but the choice would be up to the user.

In addition, the device might be capable of fixedly holding the linking device. In this manner the bending device can use another means to advance the linking device to the next bending location based on the continuous or click stopped measuring means. By fixedly holding the linking device, the measurements can be made accurately from a specific starting point adding a new starting point after each bend or using the original starting point. For example, a bend could be put at one centimeter and 3 centimeters from a starting point. In another embodiment, a bend is at the starting point and the next bend a fixed distance from the starting point. In another embodiment, by holding the linking device the linking device could be advanced based on ruled markings on the linking device instead of ruled markings on the bending device. Where on the bending device, there could be regular stop positions that are fixed or in the alternative, continuous adjustment of distance.

In general, one of the embodiments of the present invention is the process for producing bends in a surgical linking device which is comprised of two separate processes linked to each other. The first process is the digital process for determining the spatial parameters of one or more bends. The second process is the manual process of shaping a surgical linking device that applies the location, angle and rotation parameters determined in the first process. The complete linking of these two processes is facilitated by the introduction of the novel device of the present invention. The link can be the surgeon or other individual who takes the computer output and applies the result manually to the linking device.

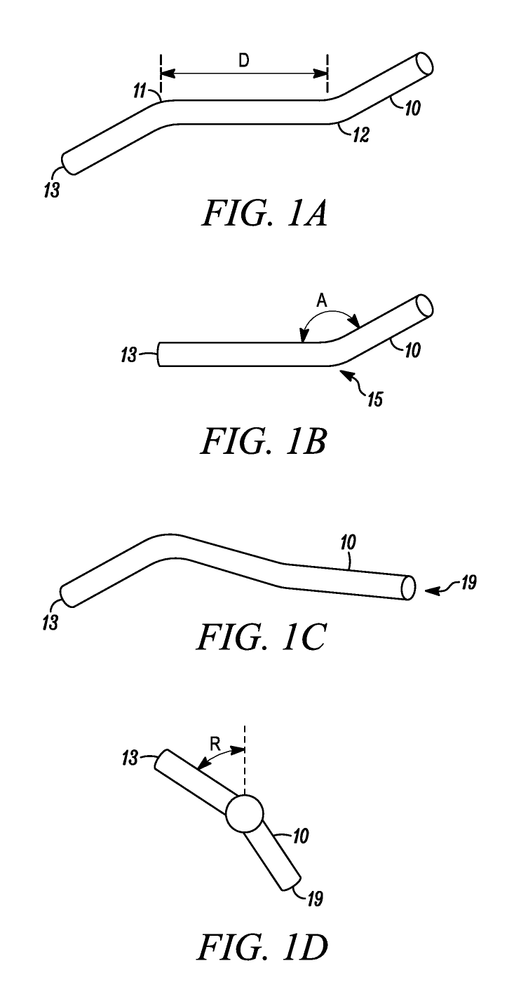

Now referring to the figures, FIGS. 1a through 1d depict various bends in a surgical rod linking device. FIG. 1a depicts a rod with a first bend 11 and second bend 12. This depiction has the rod 10 lying in one plane and the distance between bend 11 and bend 12 is shown as D. By indicating a distance D from bend 11, one can obtain the location of the second bend 12. The starting point for measurement could be either from point 13, the first rod end or bend 11. The starting point for bend location can stay with the original point for subsequent bend location determinations or can move with each bend location determination. So for example, Bend 12 could be the starting point for the next bend location determination. In FIG. 1b, a single bend 15 is shown with an angle A. The angle A is the second determined parameter of the present invention. FIGS. 1c and 1d depict a bent rod with at least one bend that has been rotated R degrees from the initial plane of the rod. Second end 19 is also depicted and in FIG. 1d the view is head on to the middle section of the bent rod 10. While a surgical rod 10 is depicted for clarity, a surgical plate or other surgical linking device could also be oriented and bent or shaped in a similar manner.

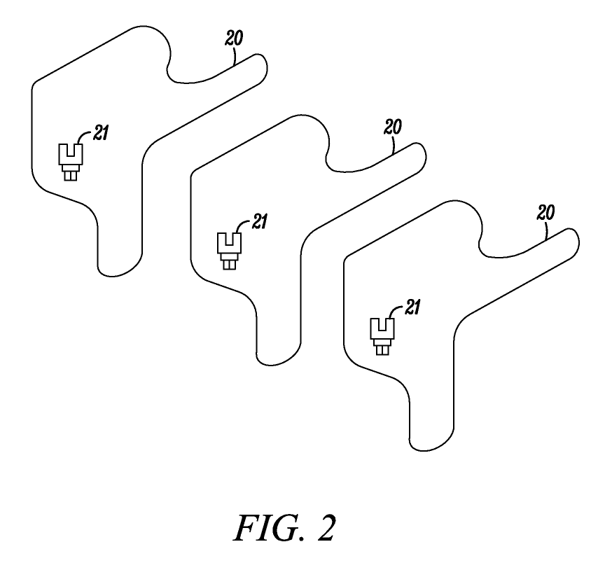

FIG. 2 depicts body structure vertebrae 20 laid out in perspective view. Each vertebrae 20 has had attachment means, vertebral screw 21 installed for the purpose of installing a surgical rod. Note that while normally rods are installed in pairs one set of screws 21 is shown for simplicity sake.

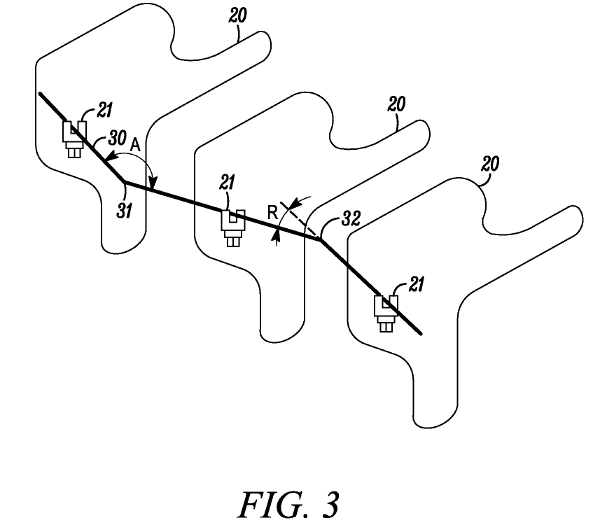

FIG. 3 depicts a bent surgical rod 30 which has been attached to the attachment means 21. Also depicted is bend angle A and rotation angle R which the rod has been bent to accommodate the positions of the attachment screws 21.

FIG. 4 is a rotation gauge 40 which may be fitted on the end of or around a surgical linking device, for example, the rod 10 depicted in FIG. 1. The rod 10 fits into hole 41 and then if the rod is rotated to the degree markings 42, a rotational bend of a selected angle can be achieved. This device could be fixedly attached to a bending device as further taught herein.

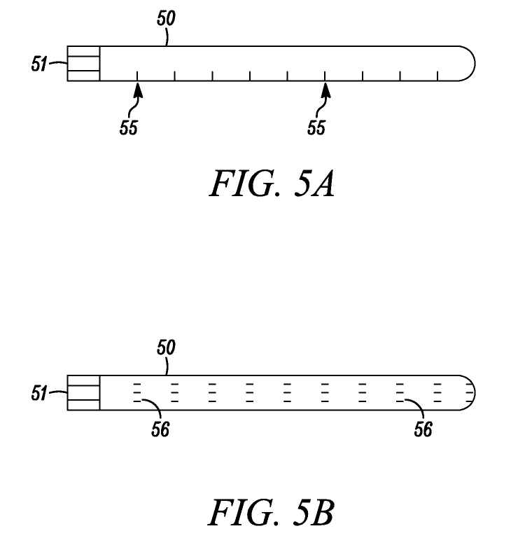

In FIGS. 5a and 5b surgical linking rods 50 are shown. These rods are normally cylindrical but first end 51 is squared off to accommodate a tool or grabbing means or the like. the gauge 40 from FIG. 4 could also be attached to this end. These surgical linking rods 50 also show either distance markings 55 to indicate the distance for a bend location. In the case of FIG. 5b rotational markings 56 are available not only for distance measurements but for rotational measurements as well.

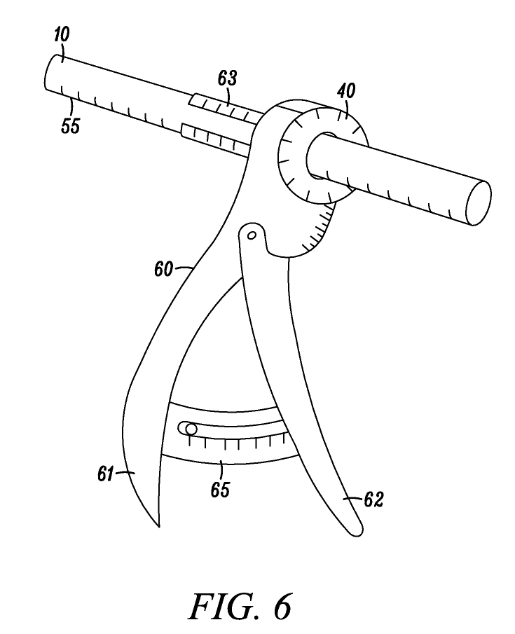

FIG. 6 depicts a simple hand bending device 60. By squeezing handles 61 and 62, rod 10 can be bent around a fulcrum (not seen). The rod is not held in place but the rod 10 is moved and by matching distance markings 63 on device 60 with rod distance markings 55 a clear location on the rod 10 can be determined. Rotational gauge 40 is installed and by manually rotating the rod 10 one can obtain a desired rotation. While the rotation is marked in intervals, this embodiment allows free rotation of rod 10 thus infinite rotational angle. The bend angle is measured by angle gauge 65. Angle gauge 65 measures the angle based on how close the handles 61 and 62 approach each other during the operation of bending rod 10.

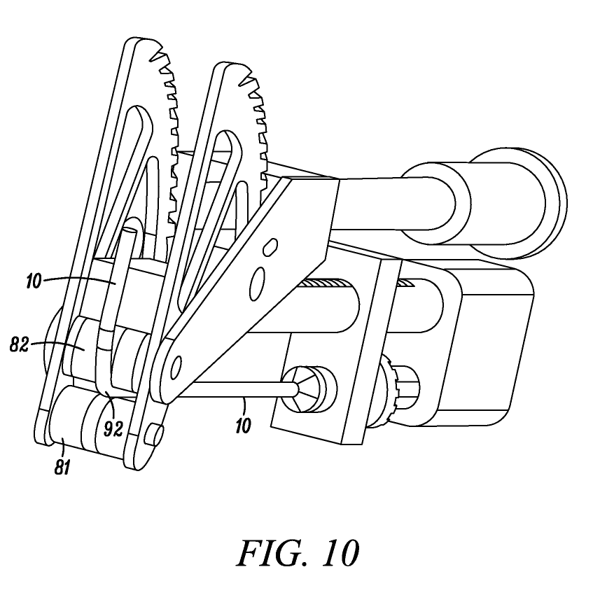

FIG. 7 is a perspective view of a more detailed bending device 70 with less manual manipulation of the rod 10. A first lever 71 is shown as is lever handle 73 designed for grabbing the lever 71 manually. Likewise, lever 72 is shown with handle grip 74. Grip 74 has rod pass through 78 so that an infinitely long rod 10 can be used with this particular handle as well as steady the rod during the bending process with bender 70. The user of the device grabs both handles and opens the handles to bend the particular surgical rod 10 by picking an angle on the angle gauge and closing the handles 71 and 72 together. The device in other embodiments could be produced to bend the rod during the handle opening movement as well. The rod 10 moves through mandrel 80 and in between moving die 81 and fixed die 82. A better view of the dies is in FIG. 10. The surgical rod is bent between the two dies 81 and 82. Gauges on the device allow the user to manipulate the surgical 10 rod in order to determine bend position, bend angle and bend rotation. The surgical rod 10 is held in place by collet 75. By sliding slide block 76, along handle 72, the surgical rod 10 can be moved proximally and distally in the bending device 70. Position is measured by click stops 77 at regular intervals along handle 72. Each click stop 77 is a measured distance along the handle 72 and thus moving a specific number of click stops 77 gives one a precise location for the location of a surgical rod 10 bend.

The bend angle is measured by using angle gauge 85. Gauge 85 has ratchet teeth 86 spaced at regular intervals. Each ratchet stop represents five degrees of bend angle. Thus the user can bend a surgical rod 10 in five degree increments with the particular bend angle gauge 85 as the handles 71 and 72 are opened and closed. The bend rotation is controlled by collet knob 90. By rotating collet knob 90 either clockwise or counterclockwise the user can set a particular rotation angle. The collet knob 90 is marked with regular interval notches 91 but this particular embodiment is continuously turn able and thus has infinite settings. Once a user turns knob 90 the user can set the knob 90 at a particular marking 91 or in between or the like to determine a particular angle rotation to a high degree of accuracy.

In this particular embodiment, once the rod 10 is locked in place with collet 75 if there is enough room on the lever 72 to move the slider 76 distally or proximally then the rod 10 can remain fixedly attached to collet 75. Should a longer area need to be bent, then the rod 10 can be unlocked moved and relocked and measurements start from the new position. Merely adding the positions together using the information supplied by the computer output would be an easy task with the present invention.

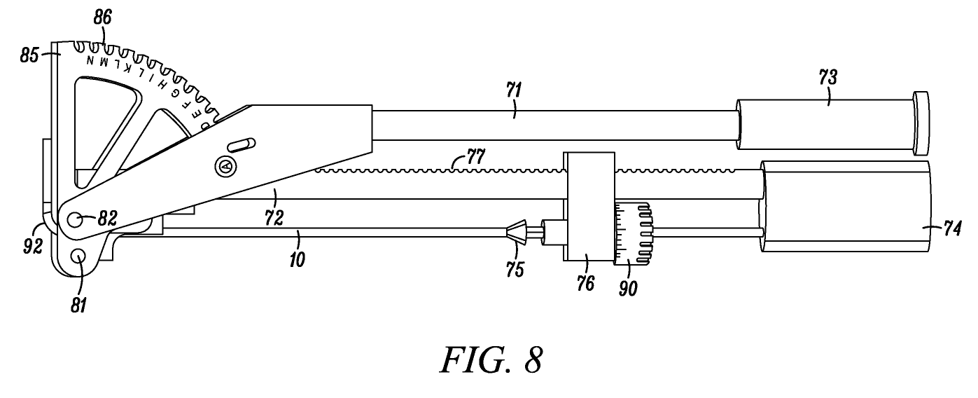

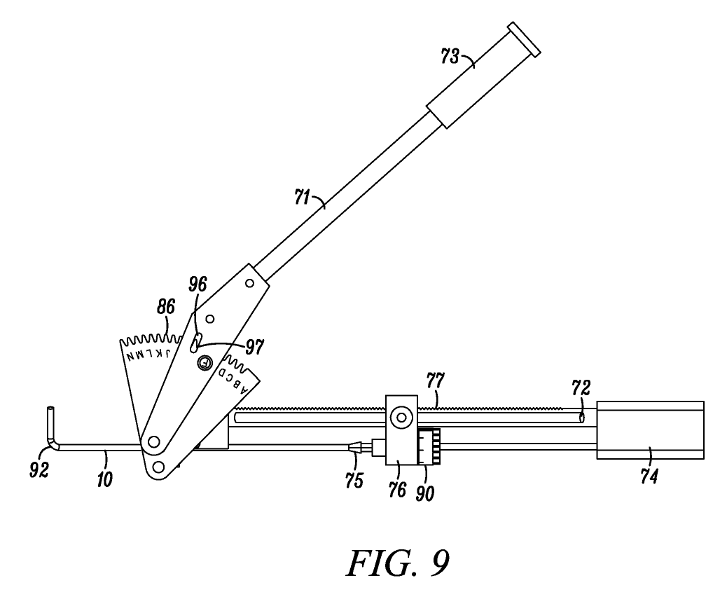

FIG. 8 depicts the bending device 70 in a side view. In this view one can clearly see the rod 10 has bend 92. FIG. 9 shows a side view wherein handle 71 is open in preparation of making a second bend in rod 10. Bend gauge window 96 shows bend angle pin 97 which has engaged 2 teeth 86 in preparation for placing the second bend. As can be seen in this view the rod 10 has moved distally since slider 76 is in a more distal position than shown in FIGS. 7 and 8. First bend 92 has moved distally as well and upon closing of levers 71 and 72 a second bend will be placed in rod 10.

FIG. 10 shows a head on view of the device 70. In this view, the rod 10 can clearly be seen in bent position between moving die 81 and fixed die 82. The moving die 81 allows for free movement of rod 10 and the fixed die 82 allows for relatively easy bending of rod 10.

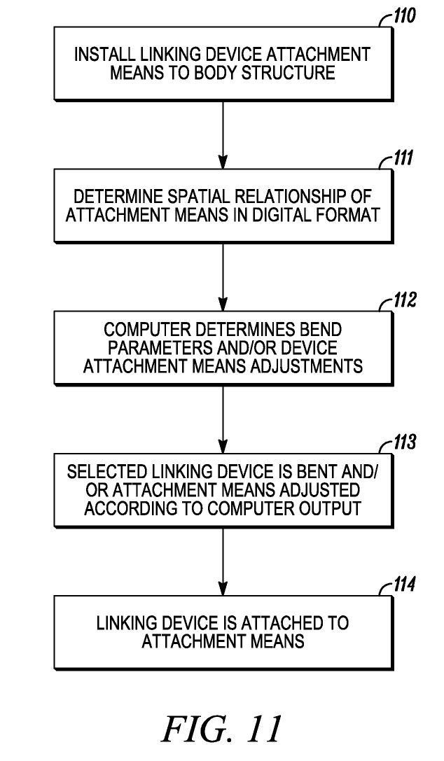

FIG. 11 depicts a flow chart of a particular embodiment of the operation of the computer means in combination with the device of the present invention. The first process is the installation of a linking device attachment means to a body structure 110. In other embodiments, for example for use with a surgical plate, the first step is to determine the surface spatial relationship of the bony structure and then using that spatial information to determine the shape of the surgical plate. Once the plate is placed on the bony structure attachment means are positioned through the plate and into the bony structure. The linking device such as screws for use with surgical rods, which to some degree adjustable then determines where the linking device will be positioned. The next step is the determination of the spatial relationship of the attachment means into a digital format 111. This is done not only taking into account the position of attachment, but also taken into consideration is any body structures which may intervene in the process. It would not be useful if say a part of the vertebrae were in the way of a particular bend solution because the resulting bent rod would not fit the attachment points because of body structure interference. One skilled in the art could easily make the appropriate adjustments to the computer calculation based on the disclosure herein. Next, the computer with the possession of the digital format determines the bend parameters and or the device attachment means adjustments 112. This could also include the selection of a particular linking device, the size it needs to be, or to select from a list of pre-bent linking devices. Once a linking device is selected from the computer output parameters, the linking device is then, if necessary bent or shaped and or the attachment means adjusted 113. After the appropriate bends have been made the linking device, it is attached to the attachment means 114.

The above examples and particular embodiments are not intend to limit the claims which follow. A variety of changes to the gauges, levers and the device and method of determining the shaping parameters is within the scope of the present invention.

What is claimed is:

1. A system for shaping a surgical linking device for attachment to a selected bony body structure having at least two linking device attachment means comprising:
a) a means for determining the relative spatial location of at least one of the attachment means and the bony structure;
b) a means for converting the relative spatial location into a digital format;
c) a computer capable of receiving the digital format in b) and using the relative spatial location to determine one or more shape locations in the surgical linking device, each shape location having one or more of a shape angle and shape rotation at each one or more shape locations such that shaping of the surgical linking device will enable the surgical linking device to attach to the bony body structure using the attachment means; and
d) a means for delivering the determined shape information to a computer output.

2. A system according to claim 1 wherein the surgical linking device is selected from the group comprising surgical plate and surgical rod.

3. A system according to claim 1 wherein the means for determining the relative spatial location and means for converting the location into a digital format is a three dimensional digitizer.

4. A system according to claim 1 wherein the means for determining the relative spatial location is a photographic means.

5. A system according to claim 1 wherein the output is a paper means.

6. A system according to claim 1 wherein the output is to a GUI.

7. A system according to claim 1 which further comprises a means for taking the computer output and shaping the surgical linking device at the one or more determined locations and having one or more of a determined shape angle and shape rotation.

8. A system according to claim 7 wherein the shaping is done by bending.

9. A system according to claim 8 wherein the device is bent manually.

10. A system according to claim 9 wherein a manual bending device is used to bend the linking device wherein the bending device is capable of determining at least one of a bend location, bend angle and bend rotation.

11. A system according to claim 8 wherein the at least one determined bend rotation is zero degrees.

12. A system according to claim 8 wherein the bend location on the linking device is determined by using ruled markings on the device.

13. A system according to claim 1 wherein the at least two attachment means are attached to the bony structure prior to the spatial location determination.

14. A system according to claim 1 which further comprises a means for the computer to determine if adjusting the position of or addition to at least one attachment means would result in eliminating one or more shaping locations.

15. A system according to claim 1 which further determines the size of the surgical linking device.

16. A system according to claim 1 wherein the attachment means are designed to be attached to the bony structure after the linking device is shaped.

17. A surgical method for installing a surgical linking device on a selected bony body structure comprising:
a) placing at least two linking device attachment means on the bony body structure at desired locations;
b) digitally determining the relative spatial location of at least one of the bony structure and the attachment means;
c) transferring the digitized information to a computer which determines information of one or more of
i. one or more of the location, angle and rotation of shapes in a selected surgical linking device that could be made in order for the linking device to be attached to the bony structure using the attachment means; and
ii. one or more adjustments to the position of or addition to the attachment means that could be made so that a selected preformed, partially preformed or a minimally shaped surgical linking device can be attached to the bony structure with the attachment means;
d) delivering the computer determined information to a computer output;
e) using the information from the computer output to perform one or more of
i. selecting a preformed or partially preformed surgical linking device;
ii. shaping a surgical linking device with a device that measures one or more of the shape location, shape angle and shape rotation; and
iii. adjusting the position of or adding to the attachment means;
f) attaching the surgical linking device to the attachment means.

18. A surgical method according to claim 17 wherein the surgical linking device is selected from the group comprising surgical plates and surgical rods.

19. A surgical method according to claim 17 wherein the shaping is bending.

20. A surgical method according to claim 17 wherein the attachment means are installed before the relative spatial location is determined.

21. A surgical method according to claim 17 wherein the desired location of the attachment means is determined by the computer analyzing the relative special location information of the bony structure.

22. A surgical method according to claim 21 wherein the attachment means are attached to the bony structure after the linking device is shaped.

23. A surgical method according to claim 17 wherein the relative spatial relationship is determined by a three dimensional digitizer.

24. A surgical method according to claim 17 wherein the linking device is bent and the attachment means position is adjusted.

25. A surgical method according to claim 17 wherein the surgical linking device is manually bent.

26. A surgical system according to claim 17 wherein the surgical linking device is preformed and then has one or more bends placed in the device.

27. A surgical system according to claim 17 which further determines the size of the surgical linking device to be selected.

28. A surgical system according to claim 17 which further comprises attaching the linking device such that it is designed to alter the position of or the shape of the selected bony body structure.

29. A device for manually bending a surgical linking device comprising:
a) a manually operated lever for bending the linking device; and
b) at least 2 bend measuring means selected from the group comprising, bend position measuring means, bend angle measuring means and bend rotation measuring means.

30. A device according to claim 29 wherein there are a pair of levers for bending the device.

31. A device according to claim 29 wherein each lever has a handle portion.

32. A device according to claim 29 which has a gauge for determining the angle that a device is bent.

33. A device according to claim 29 wherein the bend measuring means measures in a non-continuous manner.

34. A device according to claim 29 wherein the bend measuring means measures in a continuous manner.

35. A device according to claim 29 which has a gauge for measuring the rotation of a bend.

36. A device according to claim 29 which has a means for fixedly holding the attachment device while it is bent.

37. A device according to claim 29 wherein the device can use ruled markings on the attachment device to determine where a bend is to be placed.

38. A device according to claim 29 wherein the device comprises a means for advancing the linking device to a selected bend location.

39. A device according to claim 29 which has a means for establishing a starting point on the attachment device for use of the bend measuring means to measure to a bend location.

40. A device according to claim 29 comprising:
a) a fulcrum means;
b) pair of levers for bending the attachment device around the fulcrum means and having a gauge to determine a selected angle of bend; and
c) a gauge for measuring the rotation of the device prior to bending an attachment device.

41. A device according to claim 40 which further comprises a means for holding a linking device.

42. A device according to claim 40 which further comprises a measured means for advancing the linking device to the location of the bend.

43. A device according to claim 40 wherein the means for measuring the location of a bend is a measurement position on the bending device which measures against ruled markings on the linking device.

44. A device according to claim 40 wherein the means for measuring the location of a bend is based on ruled markings on the bending device.

45. A device for determining the rotation for placing a bend in a surgical linking device comprising:
a) a circular gauge indicating the degrees of rotation; and
b) a means for positioning the device on the surgical linking device or on a means for bending the linking device such that the gauge aligns with any bends in the linking device.

46. A device according to claim 45 which is capable of being attached to a device for bending the attachment device.

47. A device according to claim 45 which is capable of being attached to the attachment device.

48. A device according to claim 45 which has fixed stop positions.

49. A device according to claim 45 which has continuous stop positions.

50. A means for determining the selection of a preformed surgical linking device for use in attaching to a selected bony body structure having at least two linking device attachment means comprising:
a) a means for determining the relative spatial location of each attachment means;
b) a means for converting the relative spatial location into a digital format;
c) a plurality of preformed surgical linking devices;
d) a computer having selected spatial information about the preformed linking devices wherein the computer is capable of receiving the digital format in b) and using the digital format to determine if one of the preformed surgical linking devices fits the attachment means and if there is none that fit, if one or more attachment means could be adjusted in relative location such that one of the preformed surgical linking devices could be selected and fit the attachment means; and
e) a means for delivering the determined attachment means adjustments and selected preformed linking device to a computer output.

51. A means according to claim 50 wherein the linking devices is selected from the group comprising surgical plate and surgical rod.

52. A method for placing multiple bends with 6 degrees of freedom in a surgical linking device comprising:
a) establishing a starting point on the device;
b) holding the device relative to the starting point;
c) moving the device and measuring away from the starting point to establish a second point on the device for placing a bend with 6 degrees of freedom; and
d) repeating steps b) and c) using either the starting point or the second point to hold until the multiple bends are completed.

53. A process for producing one or more shapes in a surgical linking device comprising:
a) a digital process for determining the desired spatial parameters of the shapes to be produced; and
b) a manual shaping process linked to the digital process wherein the shaping process applies the spatial parameters to the surgical attachment device.

54. A process according to claim 53 wherein the spatial parameters are selected from the group comprising linking device size, attachment means location, shape location, shape angle and shape rotation.
